(19) United States
(12) Patent Application Publication
(10) Pub. No.: US 2025/0235666 A1
(43) Pub. Date: Jul. 24, 2025

(54) MEDICAL SYSTEMS, DEVICES, AND RELATED METHODS FOR STEERING OR DEFLECTING A PORTION OF A MEDICAL DEVICE

(71) Applicant: Boston Scientific Scimed, Inc., Maple Grove, MN (US)

(72) Inventors: Nathan STENGER, Minneapolis, MN (US); Jason John MATTESON, JR., Beldenville, WI (US); Mark Phillip OLSON, Saint Anthony, MN (US)

(73) Assignee: Boston Scientific Scimed, Inc., Maple Grove, MN (US)

(21) Appl. No.: 19/032,638

(22) Filed: Jan. 21, 2025

Related U.S. Application Data
(60) Provisional application No. 63/624,022, filed on Jan. 23, 2024.

(51) Int. Cl. A61M 25/01; A61M 25/00

(52) U.S. Cl. CPC A61M 25/0136; A61M 25/0074; A61M 25/0147; A61M 2025/015

(57) ABSTRACT

Medical devices and related methods are described. The medical device includes a handle including a movable body, a shaft extending from a distal end of the handle, a first fitting coupled to the shaft, and a second fitting distal to the first fitting and concentrically coupled to the shaft. The second fitting includes a first hole and a second hole. The first hole receives a portion of the shaft, and the second hole is aligned with the first fitting. A third fitting surrounds the shaft, the first fitting, and the second fitting. The third fitting includes a distal face, a third hole, and a fourth hole. The third hole receives a distal portion of the shaft, and the fourth hole is aligned with the second hole and a wire. A proximal portion of the wire is coupled to the first fitting.

### CROSS-REFERENCE TO RELATED APPLICATIONS

This application claims the benefit of priority under 35 U.S.C. § 119 from U.S. Provisional Application No. 63/624,022, filed Jan. 23, 2024, which is incorporated by reference herein in its entirety.

### TECHNICAL FIELD

Various aspects of this disclosure relate generally to systems, devices, and related methods useful in medical procedures. More specifically, this disclosure relates to systems, devices, and methods for steering or deflecting a portion (e.g., a distal portion) of a medical device.

### BACKGROUND

Steerable medical devices typically utilize one or more pull wires to steer or deflect a distal portion of the medical device, for example, through anatomical paths within the human body, such as the stomach. A tensioned pull wire typically bends in a manner that follows a path of least resistance within a lumen of the human body. This tendency may cause a catheter to move in a whip-like manner at a distal end of the catheter, as torque accumulates in proximal portions of the catheter and subsequently moves to the distal end of the catheter. This whipping movement may lead to an increased likelihood of a pull wire breaking and/or failed medical devices. The general trend towards minimally invasive procedures and correspondingly smaller (i.e., outer diameter) catheters with lower tensile strength pull wires and torque responses further exacerbates the likelihood of whipping and/or breakage during a medical procedure.

The systems, device, and methods of this disclosure may rectify one or more of the deficiencies described above or address other aspects of the art.

### SUMMARY

Examples of the disclosure relate to, among other things, medical systems, devices, and methods configured for steering or deflecting a catheter or other medical device through anatomical paths of the human body to perform a medical procedure therein. Each of the examples disclosed herein may include one or more of the features described in connection with any of the other disclosed examples.

For example, this disclosure includes a medical device that may include a handle and a shaft extending from a distal end of the handle. The handle may include a movable body. The medical device may include a first fitting coupled to the shaft, and a second fitting distal to the first fitting and concentrically coupled to the shaft The second fitting may comprise a first hole and a second hole. The first hole may receive a portion of the shaft, and the second hole may be aligned with the first fitting. The medical device may further comprise a third fitting surrounding the shaft, the first fitting, and the second fitting. The third fitting may comprise a distal face, a third hole, and a fourth hole. The third hole may receive a distal portion of the shaft, and the fourth hole may be aligned with the second hole and a wire. A proximal portion of the wire may be coupled to the first fitting. A portion of the wire may extend through the second hole, and a distal portion of the wire may extend through the fourth hole.

Any of the medical devices disclosed herein may include any of the following features. The medical device may further comprise a fourth fitting, proximal to the third fitting, concentrically surrounding the shaft, the first fitting, and the second fitting. The fourth fitting may comprise a proximal face. The proximal face may comprise a fifth hole that movably receives a portion of the shaft. The fourth fitting may be fixed to the third fitting. The medical device may further comprise a rod coupled to the second fitting and positioned opposite the second hole. The rod may extend distally of the second fitting. The medical device may further comprise an articulable portion coupled to the distal face. The wire may be fixedly coupled to a portion of the articulable portion. Rotation of the handle may rotate the shaft to rotate the articulable portion. Proximal movement of the shaft may move the wire independent of the third fitting and the fourth fitting, and may transition the articulable portion between at least a first configuration and a second configuration. Distal movement of the shaft may move the wire independent of the third fitting and the fourth fitting, and may transition the articulable portion between at least the second configuration to the first configuration. Rotational movement of the shaft relative to the third fitting and the fourth fitting may be limited by the rod interacting with a rod-receiving hole disposed on the distal face. Proximal movement of the shaft may be limited by the first fitting interacting with the proximal face of fourth fitting. The shaft may comprise a hypodermic tube. The third fitting may concentrically surround the shaft. The proximal face may comprise a taper from an outer diameter of the distal face to a diameter approximately equal to the shaft. The wire may have an outer diameter less than or equal to 0.008 inches. The wire may comprise one of (i) a cable, or (ii) a solid wire.

This disclosure also includes a medical device that may include a handle. The handle may include a moveable body. The medical device may further include a shaft extending from a distal end of the handle. The shaft may be coupled to the movable body. The medical device may further include a first fitting coupled to the shaft. The medical device may further include a second fitting distal to the first fitting and coupled to the shaft. The second fitting may comprise a first hole and a second hole. The first hole may receive a portion of the shaft, and the second hole may be circumferentially aligned with the first fitting. The medical device may further comprise a third fitting surrounding the shaft, the first fitting, and the second fitting. The third fitting may comprise a distal face. The distal face may comprise a third hole that may receive the shaft, and the distal face may also comprise a fourth hole that may be circumferentially aligned with the second hole. Furthermore, the distal face may include a fifth hole that may be opposite the fourth hole. The medical device may further comprise a member coupled to the second fitting and positioned opposite the second hole. The member may be circumferentially aligned with the fifth hole. The member extending distally from the second fitting. The medical device may further include a wire. The wire may be coupled to and extend from the first fitting, through the second hole, and through the fourth hole.

The medical device may include one or more of the following features. The medical device may further comprise a fourth fitting, proximal to the third fitting, surrounding the shaft, the first fitting, the second fitting. The fourth fitting may comprise a proximal face. The proximal face may comprise a sixth hole that receives the shaft. The fourth fitting may be fixed to the third fitting. The medical device may further comprise an articulable portion coupled to the distal face and fixed to the wire. Rotation of the handle may rotate the shaft to rotate the articulable portion. Longitudinal movement of the shaft may translate the member through the third fitting. Distal movement of the shaft (1) may move the wire independent of the third fitting and the fourth fitting, and (2) may transition the articulable portion between at least a first configuration and a second configuration. Proximal movement of the shaft (1) may move the wire independent of the third fitting and the fourth fitting and (2) may transition the articulable portion between at least a first configuration and a second configuration.

The present disclosure also includes a medical device comprising a handle. The handle may include a movable body. The medical device further includes a shaft extending from a distal end of the handle and coupled to the movable body. The medical device also includes a first fitting coupled to the shaft, a second fitting distal to the first fitting and coupled to the shaft. The second fitting may include a first hole and a second hole. The first hole may receive a portion of the shaft, and the second hole may be aligned with the first fitting. The medical device may further comprise a third fitting surrounding the shaft, the first fitting, the second fitting, the third fitting comprising a distal face. The distal face may comprise a third hole that receives a portion of the shaft and a fourth hole aligned with the second hole. The shaft may be longitudinally movable relative to the third fitting. The medical device may further comprise a wire extending from the first fitting, through the second hole and through the fourth hole.

The medical device may include one or more of the following features. The medical device may further comprise a fourth fitting, proximal to the third fitting and surrounding the shaft, the first fitting, and the second fitting. The shaft may be longitudinally movable relative to the fourth fitting. The fourth fitting may comprise a proximal face. The proximal face may comprise a sixth hole that receives a portion of the shaft.

### BRIEF DESCRIPTION OF THE DRAWINGS

The accompanying drawings, which are incorporated in and constitute a part of this specification, illustrate various exemplary embodiments and together with the description, serve to explain the principles of the disclosure.

FIG. 1 depicts an exemplary medical device, according to aspects of this disclosure.

FIG. 2A depicts a perspective view of a portion of the exemplary medical device of FIG. 1, and FIG. 2B depicts a side view of the portion of the exemplary medical device of FIG. 1.

FIG. 3A depicts a distal portion of the medical device of FIG. 1 in an unarticulated or undeflected state, and FIG. 3B depicts the distal portion of the medical device of FIG. 1 in an articulated or deflected state.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The drawings may include arrows labeled “P” and “D,” indicating proximal and distal directions, respectively. The term “approximately,” or like terms (e.g., “substantially”), includes values +/−10% of a stated value.

Reference is now made in detail to examples of this disclosure, aspects of which are illustrated in the accompanying drawings. Wherever possible, the same reference numbers will be used throughout the drawings to refer to the same or like parts.

Aspects of this disclosure seek to improve configurations of steering components to increase pull wire tensile strength, reduce rates of pull wire breakage, increase pull wire torque, reduce the need to remove and reintroduce a medical device into a subject's body, help treat tissue within the subject, reduce overall medical device size and/or cost, and/or reduce overall procedure time, among other aspects. In an example, aspects of the disclosure may provide a user (e.g., physician, medical technician, or other medical service provider) with the ability to steer a medical device with increased responsiveness and/or handling, for example, through anatomical paths of a patient.

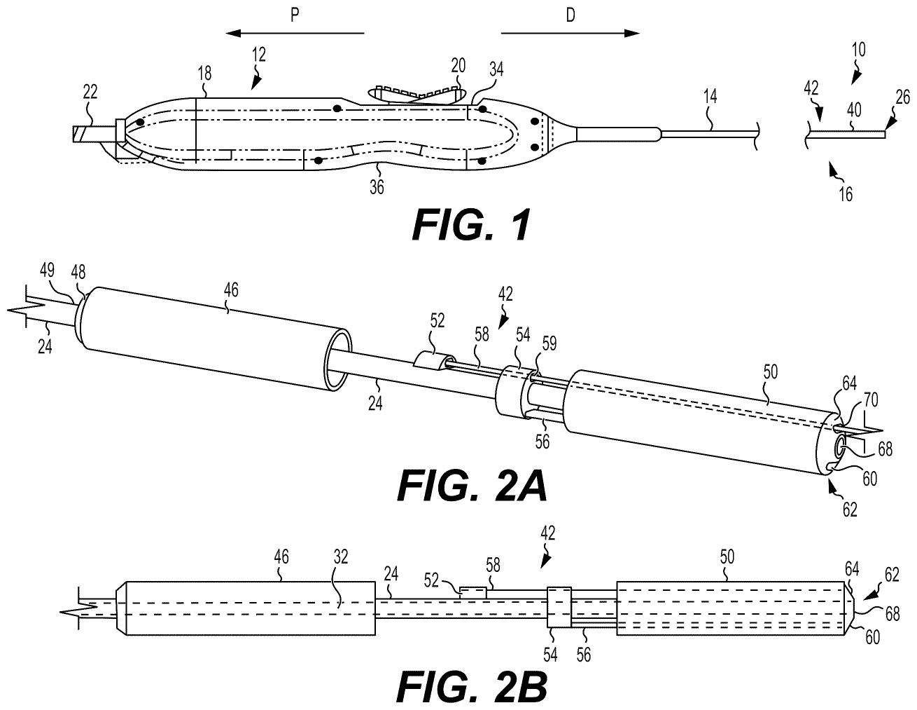

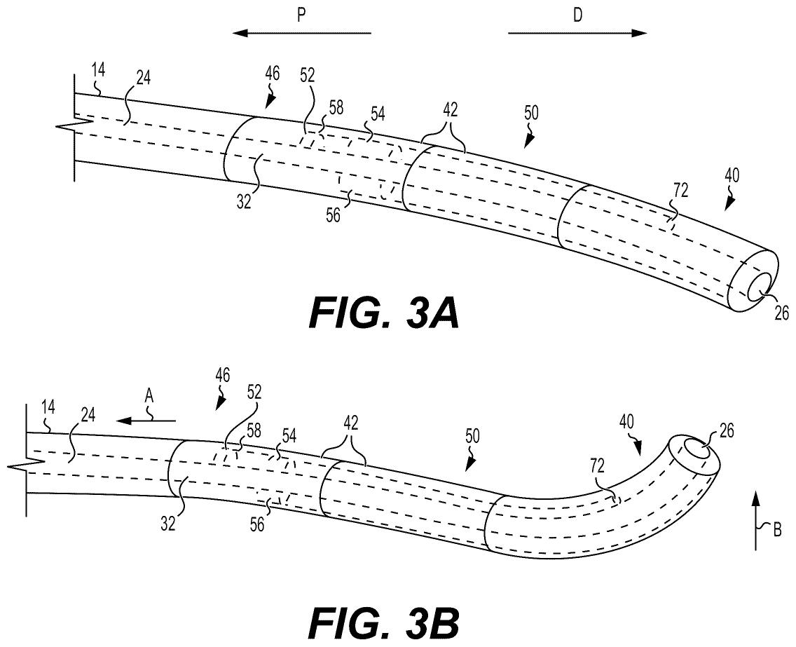

FIG. 1 depicts a medical device 10 that includes a handle 12 and a sheath or an insertion portion 14, which includes a distal end 16. Handle 12 may include a main body 18 and a movable body 20. Handle 12 also may include a port 22 (e.g., a biopsy port) configured to receive fluid and/or another medical device, for example, including an end effector. Distal end 16 includes distal opening 26 (FIGS. 3A and 3B), which may be fluidly connected to port 22, for example, via a lumen or working channel that extends through handle 12 and insertion portion 14. Insertion portion 14 includes a steerable section or steerable portion 40, for example, at a position proximal to distal end 16. Additionally, insertion portion 14 includes a control portion or a torque delivery portion 42, for example, proximal of steerable portion 40. As discussed in detail below, movable body 20 is coupled (e.g., directly or indirectly coupled) to an internal shaft 24 (FIGS. 2A and 2B). Additionally, movement of movable body 20 may control movement of internal shaft 24, for example, to control one or more aspects of steerable portion 40 and/or torque delivery portion 42 to articulate or deflect a portion of insertion portion 14. Although not shown in FIG. 1, it should be understood that internal shaft 24 may be disposed (e.g., longitudinally movably disposed) within a portion of handle 12 and at least a portion of insertion portion 14.

Medical device 10 may be inserted into a body lumen of a subject, either through an insertion device (not shown) or alone, such that at least a portion of insertion portion 14 may be within the subject, while handle 12 may remain outside of the subject. Distal end 16 may be positioned at or adjacent to a target site within the subject. From outside of the subject, a user can manipulate handle 12. Movement of movable body 20 relative to main body 18 in a first direction (e.g., the proximal direction) may also move internal shaft 24 in the first direction (e.g., proximally). As discussed in more detail below, internal shaft 24 may be coupled (e.g., directly or indirectly coupled) to one or more pull wires, for example, in torque delivery portion 42. Distal ends of the one or more pull wires are then coupled to one or more portions of steerable portion 40. In these aspects, proximal movement of internal shaft 24 may also proximally retract the one or more pull wires, for example, to deflect or articulate steerable portion 40 (e.g., to bend in one or more directions, for example, radially outward).

Handle 12 may be coupled to a fluid source (not shown) via port 22. Port 22 may be in fluid communication with distal opening 26 via an internal lumen 32 (FIG. 2B), which may extend through handle 12 and insertion portion 14. For example, a portion of internal lumen 32 that extends through insertion portion may be formed by an internal lumen or channel within internal shaft 24. In some aspects, internal shaft 24 may be a hypodermic tube. In one aspect, internal lumen 32 may extend longitudinally through main body 18 of handle 12 and insertion portion 14 (e.g., through internal shaft 24) to fluidly connect port 22 to distal opening 26. Port 22 may be positioned on a proximal portion of main body 18, for example, a proximal end of main body 18. Alternatively, port 22 may be positioned on a distal or central portion of main body 18. Moreover, port 22 may include a one-way valve, a luer, a seal, threading, and/or any appropriate element to help maintain a secure connection between handle 12 and the fluid source, minimize or prevent back-flow (e.g., fluid flowing proximally out of port 22), and/or minimize or prevent leakage. In one example, a one-way valve may include an outer housing containing an inner elastomeric and/or gelatinous sealing member (not shown).

Handle 12 may control the movement and/or positioning of internal shaft 24, for example, relative to the distal end 16 of internal shaft 24. For example, main body 18 may include a slot 34, and movable body 20 may be slidably positioned within slot 34. Slot 34 may define a range of movement (e.g., longitudinal movement) of movable body 20, which may, in turn, define a range of movement (e.g., longitudinal movement) of internal shaft 24. Main body 18 may be configured to be held by a user's hand, and movable body 20 may be configured to be controlled by the movement of the user's thumb. For example, a side of main body 18 opposite to movable body 20 may include one or more contours 36, which may help the user grip main body 18. Movable body 20 may be lockable in one or more positions relative to main body 18, and/or may be spring-biased in a direction (e.g., toward a distally extended position). As mentioned, movable body 20 may be coupled to internal shaft 24. For example, movable body 20 may extend within a portion of main body 18 (e.g., via slot 34), and may be directly coupled to a proximal portion of internal shaft 24. Alternatively, main body 18 may be indirectly coupled to internal shaft 24. In these aspects, movement of movable body 20 may control the movement of internal shaft 24.

As shown in FIG. 1, insertion portion 14 extends from a distal portion of main body 18 to distal end 16. Insertion portion 14 may include a sheath that may surround or otherwise form at least a portion of distal opening 26. Insertion portion 14 may be a tube or a sheath that surrounds at least a portion of one or more lumens (e.g., internal lumen 32) and internal shaft 24. In another aspect, insertion portion 14 may be an extrusion that includes one or more lumens extending from handle 12 to distal end 16.

FIGS. 2A and 2B depict additional aspects of torque delivery portion 42 of medical device 10. FIG. 2A depicts a perspective view of torque delivery portion 42 of the exemplary medical device 10 of FIG. 1 in a partially exploded configuration, and FIG. 2B depicts a side view of torque delivery portion 42 of the exemplary medical device 10 of FIG. 1 in the partially exploded configuration. Torque delivery portion 42 may be proximal to and coupled to steerable portion 40 (shown in FIGS. 1 and 3A-3B).

Referring now to FIG. 2A, torque delivery portion 42 includes a proximal housing 46 and a distal housing 50, which may both concentrically or radially surround a portion of internal shaft 24. Internal shaft 24 is movable (e.g., longitudinally movable) within proximal housing 46 and distal housing 50. Distal housing 50 may concentrically surround internal shaft 24 in a substantially similar manner to proximal housing 46. It should be understood that proximal housing 46 and distal housing 50 are depicted as separate components to illustrate internal features.

Torque delivery portion 42 may include one or more of a proximal fitting 52, a steering fitting 54, a guiding element 56, and a wire 58 Proximal housing 46 and distal housing 50 may be fixed (e.g., welded, adhered, etc.) to one another (e.g., with a distal end of proximal housing 46 coupled to a proximal end of distal housing 50). In these aspects, proximal housing 46 and distal housing 50 concentrically enclose a portion of internal shaft 24, proximal fitting 52, steering fitting 54, guiding element 56, and wire 58. Proximal housing 46 and distal housing 50 are not fixedly coupled to internal shaft 24. In these aspects, movement (e.g., longitudinal movement) of internal shaft 24 may cause internal shaft 24 to freely translate through or within both proximal housing 46 and distal housing 50.

Proximal housing 46 may feature a taper 48. Taper 48 may define a slope between an outer diameter of proximal housing 46 and a diameter approximately equal to an outer diameter of internal shaft 24. In some aspects, a portion of the sheath that defines insertion portion 14 may be coupled to (e.g., fixedly coupled to) a proximal face (e.g., taper 48) of proximal housing 46. Alternatively, although not shown, one or more portions of torque delivery portion 42 and/or steerable portion 40 may be enclosed or surrounded by a sheath to form respective portions of insertion portion 14. Proximal housing 46 may receive internal shaft 24 through a proximal opening 49.

Distal to proximal housing 46 is a proximal fitting 52. Proximal fitting 52 may be welded, adhered, crimped, or otherwise fixedly attached to internal shaft 24. One or more wires 58 may be fixed (e.g., welded, adhered, crimped, etc.) to proximal fitting 52. For example, a distal face or portion of proximal fitting 52 may include an opening to receive a proximal portion of wire 58. Wire 58 may extend distally from proximal fitting 52. Wire 58 may have an outer diameter of approximately 0.008 inches or less. As described previously, proximal fitting 52 may be positioned within proximal housing 46 and distal housing 50. As internal shaft 24 translates longitudinally (e.g., proximally or distally), proximal fitting 52 translates with internal shaft 24. As internal shaft 24 translates proximally, the proximal movement of proximal fitting 52 is limited by taper 48, which prevents further proximal longitudinal translation of proximal fitting 52. Limiting the proximal movement of proximal fitting 52 also helps to limit the proximal movement of internal shaft 24.

As mentioned above, torque delivery portion 42 includes steering fitting 54, for example, distal to proximal fitting 52. Steering fitting 54 may be welded, crimped, adhered, or otherwise fixedly attached to internal shaft 24. Steering fitting 54 may concentrically surround internal shaft 24. Steering fitting 54 may feature a steering fitting hole 59 that is approximately aligned with proximal fitting 52. Wire 58 may be disposed through steering fitting hole 59, such that wire 58 is substantially parallel with internal shaft 24. Guiding element 56 may be coupled to steering fitting 54, and may extend distally, for example, substantially parallel to the longitudinal axis of internal shaft 24. Guiding element 56 may be welded, adhered, or otherwise immovably fixed to an opposite (i.e., circumferentially) side of steering fitting 54 relative to steering fitting hole 59. Guiding element 56 may be a rigid or semi-rigid member that is disposed through a space between internal shaft 24 and an inner diameter of distal housing 50. Guiding element 56 may also be a rod.

As steering fitting 54 translates proximally with internal shaft 24, guiding element 56 translates through guiding element hole 60. Guiding element hole 60 may also be referred to a rod-receiving hole. Guiding element hole 60 may be extend through (e.g., longitudinally through) distal housing 50. Guiding element hole 60 may be aligned with guiding element 56. As internal shaft 24 translates proximally or distally along the longitudinal axis, guiding element 56 may translate within guiding element hole 60. Translation of guiding element 56 within guiding element hole 60 may help to prevent rotation of internal shaft 24 relative to proximal housing 46 and/or distal housing 50 as internal shaft 24 translates proximally and/or distally. Guiding element 56 also helps to keep elements

Distal face of distal housing 62 may include a taper 64, for example, which may be substantially similar to taper 48. Taper 64 may define a slope between an outer diameter of distal housing 50 and a diameter approximately equal to an outer diameter of internal shaft 24. Distal housing 50 may receive a distal portion of internal shaft 24, for example, through a central hole 68. Central hole 68 may extend longitudinally through distal housing 50, for example, from the proximal face to the distal face of distal housing 50. Also, distal housing 50 may also include a wire connection hole 70, which may receive a distal portion of wire 58.

Distal face of distal housing 62 features wire connection hole 70. Wire connection hole 70 may be longitudinally aligned with proximal fitting 52 and steering fitting 54, for example, so as to receive a distal portion of wire 58. Wire 58 may continue distally through wire connection hole 70 and may be welded, adhered, or otherwise fixedly coupled to a portion (e.g., an inner diameter) of steerable portion 40 (FIGS. 1, 3A, and 3B). Wire 58 may comprise a cable or a solid wire.

FIG. 2B shows a side view of torque delivery portion 42, with certain internal elements shown in dashed lines. In an example, as a user translates internal shaft 24 proximally, all components coupled to internal shaft 24 will similarly translate. For example, proximal fitting 52, wire(s) 58, steering fitting 54, and guiding element 56 may also translate with internal shaft 24. As described above, proximal fitting 52 may be connected to a portion (e.g., an inner diameter) of steerable portion 40 via wire 58. Wire 58 may be disposed longitudinally through steering fitting hole 59, a space between internal shaft 24 and an inner diameter of distal housing 50, and wire connection hole 70.

FIG. 2B also illustrates the interface between guiding element 56 and an inner diameter of distal housing 50. As internal shaft 24 translates proximally and/or distally, guiding element 56 similarly translates and engages guiding element hole 60. Guiding element 56 helps to prevent rotation of internal shaft 24 (and components coupled thereon) relative to proximal housing 46 and distal housing 50 as internal shaft 24 is moving through one or more lumens within a human body. Internal lumen 32 of internal shaft 24 may be understood to be a working channel capable of receiving one or more instruments, fluids, suction, sharps, guidewires, contrast flush, and other treatments.

Internal shaft 24 defines a portion of internal lumen 32, through which various instruments and/or fluids may be delivered to a target site within the human body, for example, as part of an endoscopy procedure. Internal lumen 32 may be connected to steerable portion 40 via central hole 68 disposed on distal face of distal housing 62, as well as wire 58. Torque delivery portion 42 and steerable portion 40 may be fixedly coupled, for example, via a concentric weld around central hole 68. Internal lumen 32 may continue distally through steerable portion 40

FIG. 3A shows proximal housing 46 and distal housing 50 in an assembled state, and with steerable portion 40 attached to distal housing 50. As shown in FIG. 3A, steerable portion 40 is in an unarticulated state. Steerable portion 40 may articulate or deflect based on force exerted by wire 58, for example, based on movement(s) of wire 58 that correspond to movement(s) of internal shaft 24. Additionally, wire 58 is depicted as being anchored to an internal portion of steerable portion 40, shown at anchor point 72.

FIG. 3B shows steerable portion 40 in an articulated state. Longitudinal movement of wire 58 may cause wire 58 to push or pull on anchor point 72 within steerable portion 40, causing steerable portion 40 to articulate. It should be understood that, in some aspects, wire 58 may be of sufficient rigidity to be pushed, as well as pulled, for example, to help articulate or deflect steerable portion 40 in an opposite direction to the direction shown in FIG. 3B. Also depicted in FIG. 3B is proximal fitting 52 having moved proximally with internal shaft 24, as compared to FIG. 3A. Rotation of insertion portion 14 (e.g., via rotation of handle 12) and guiding element 56 may help to allow for distal end 16 to deflect through a range of motion, for example, a semi-spherical range of motion.

Each of the embodiments discussed herein are exemplary. The various configurations may be utilized, for example, such that torque delivery portion 42 may increase torque delivery and steering response in micro-steerable devices, such as endoscopes. Traditional steering wires are locked into a defined position within an internal lumen of an endoscope. The exemplary torque delivery may reduce the likelihood of pull wire breakage and may help to increase torque response through anatomical curvature.

While principles of this disclosure are described herein with the reference to illustrative examples in a particular context and for particular medical procedures, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a handle, wherein the handle includes a movable body;
a shaft extending from a distal end of the handle;
a first fitting coupled to the shaft;
a second fitting distal to the first fitting and concentrically coupled to the shaft, the second fitting comprising a first hole and a second hole, wherein the first hole receives a portion of the shaft, and wherein the second hole is aligned with the first fitting;
a third fitting surrounding the shaft, the first fitting, and the second fitting, wherein the third fitting comprises a distal face, a third hole, and a fourth hole, wherein the third hole receives a distal portion of the shaft, and wherein the fourth hole is aligned with the second hole; and
a wire, wherein a proximal portion of the wire is coupled to the first fitting, wherein a portion of the wire extend through the second hole, and wherein a distal portion of the wire extends through the fourth hole.

2. The medical device of claim 1, further comprising:
a fourth fitting, proximal to the third fitting, concentrically surrounding the shaft, the first fitting, the second fitting, wherein the fourth fitting comprises a proximal face, wherein the proximal face comprises a fifth hole that movably receives a portion of the shaft.

3. The medical device of claim 2, wherein the fourth fitting is fixed to the third fitting.

4. The medical device of claim 3, further comprising:
a rod coupled to the second fitting and positioned opposite the second hole, wherein the rod extends distally of the second fitting.

5. The medical device of claim 4, further comprising:
an articulable portion coupled to the distal face, wherein the wire is fixedly coupled to a portion of the articulable portion.

6. The medical device of claim 5, wherein rotation of the handle rotates the shaft to rotate the articulable portion.

7. The medical device of claim 5, wherein proximal movement of the shaft moves the wire independent of the third fitting and the fourth fitting and transitions the articulable portion between at least a first configuration and a second configuration.

8. The medical device of claim 7, wherein distal movement of the shaft moves the wire independent of the third fitting and the fourth fitting and transitions the articulable portion between at least the second configuration to the first configuration.

9. The medical device of claim 4, wherein rotational movement of the shaft relative to the third fitting and the fourth fitting is limited by the rod interacting with a rod-receiving hole disposed on the distal face.

10. The medical device of claim 4, wherein proximal movement of the shaft is limited by the first fitting interacting with the proximal face of fourth fitting.

11. The medical device of claim 1, wherein the shaft comprises a hypodermic tube.

12. The medical device of claim 1, wherein the third fitting concentrically surrounds the shaft.

13. The medical device of claim 2, wherein the proximal face comprises a taper from an outer diameter of the distal face to a diameter approximately equal to the shaft.

14. The medical device of claim 1, wherein the wire has an outer diameter less than or equal to 0.008 inches.

15. The medical device of claim 1, wherein the wire comprises one of (i) a cable, or (ii) a solid wire.

16. A medical device, comprising:
a handle, wherein the handle includes a movable body;
a shaft extending from a distal end of the handle and coupled to the movable body;
a first fitting coupled to the shaft;
a second fitting distal to the first fitting and coupled to the shaft, the second fitting comprising a first hole and a second hole, wherein the first hole receives a portion of the shaft, and wherein the second hole is circumferentially aligned with the first fitting;
a third fitting surrounding the shaft, the first fitting, the second fitting, the third fitting comprising a distal face, wherein the distal face comprises a third hole that receives the shaft and a fourth hole circumferentially aligned with the second hole, and a fifth hole opposite the fourth hole;
a member coupled to the second fitting and positioned opposite the second hole and circumferentially aligned with the fifth hole, the member extending distally from the second fitting; and
a wire, coupled to and extending from the first fitting, through the second hole and through the fourth hole.

17. The medical device of claim 16, further comprising:
a fourth fitting, proximal to the third fitting, surrounding the shaft, the first fitting, the second fitting, wherein the fourth fitting comprises a proximal face, wherein the proximal face comprises a sixth hole that receives the shaft, wherein the fourth fitting is fixed to the third fitting; and
an articulable portion coupled to the distal face and fixed to the wire.

18. The medical device of claim 17, wherein rotation of the handle rotates the shaft to rotate the articulable portion, wherein longitudinal movement of the shaft translates the member through the third fitting,
wherein distal movement of the shaft (1) moves the wire independent of the third fitting and the fourth fitting and (2) transitions the articulable portion between at least a first configuration and a second configuration,
wherein proximal movement of the shaft (1) moves the wire independent of the third fitting and the fourth fitting and (2) transitions the articulable portion between at least a first configuration and a second configuration.

19. A medical device, comprising:
a handle, wherein the handle includes a movable body;
a shaft extending from a distal end of the handle and coupled to the movable body;
a first fitting coupled to the shaft;
a second fitting distal to the first fitting and coupled to the shaft, the second fitting comprising a first hole and a second hole, wherein the first hole receives a portion of the shaft, and wherein the second hole is aligned with the first fitting;
a third fitting surrounding the shaft, the first fitting, the second fitting, the third fitting comprising a distal face, wherein the distal face comprises a third hole that receives a portion of the shaft and a fourth hole aligned with the second hole, wherein the shaft is longitudinally movable relative to the third fitting; and
a wire extending from the first fitting, through the second hole and through the fourth hole.

20. The medical device of claim 19, further comprising:
a fourth fitting, proximal to the third fitting and surrounding the shaft, the first fitting, and the second fitting, wherein the shaft is longitudinally movable relative to the fourth fitting, wherein the fourth fitting comprises a proximal face, wherein the proximal face comprises a sixth hole that receives a portion of the shaft.
